(19) United States
(12) Patent Application Publication
(10) Pub. No.: US 2023/0128478 A1
(43) Pub. Date: Apr. 27, 2023

(54) ISOLATION AND DETECTION OF CDCP1 POSITIVE CIRCULATING TUMOR CELLS

(71) Applicant: Universitaetsklinikum Hamburg-Eppendorf, Hamburg (DE)

(72) Inventors: Sebastian Gaertner, Hamburg (DE); Kai Bartkowiak, Grosshansdorf (DE); Klaus Pantel, Hamburg (DE); Parinaz Mossahebi Mohammadi, Hamburg (DE)

(21) Appl. No.: 17/510,826

(22) Filed: Oct. 26, 2021

(51) Int. Cl. G01N 33/50; G01N 33/543; G01N 33/531

(52) U.S. Cl. CPC G01N 33/5005; G01N 33/54326; G01N 33/54346; G01N 33/531; B82Y 25/00

(57) ABSTRACT

A method for the isolation, or isolation and detection, of circulating tumor cells (CTCs) from blood or lymph, or disseminated tumor cells (DTCs) from bone marrow. CDCP1 is used as a biomarker for the isolation of CTCs or DTCs having a mesenchymal phenotype (mCTC, mDTC) or a hybrid epithelial/mesenchymal phenotype (emCTC, emDTC). Isolation can, for example, be done immunomagnetically using anti-CDCP1 antibodies coupled to magnetic particles.

### BACKGROUND OF THE INVENTION

### Field of the Invention

The invention relates to an improved method for the isolation, and optionally including detection, of circulating tumor cells (CTCs) from blood or lymph and/or disseminated tumor cells (DTCs) from bone marrow. Isolation of CTCs and/or DTCs allows for a subsequent genetic or other analysis, e.g. for developing therapies.

### Description of the Related Art

CTCs are tumor cells that detach from a primary tumor, enter the circulation (blood or lymphatic system) and reach secondary sites in the body. When CTCs have settled down at secondary sites, they are called disseminated tumor cells (DTCs), which are the precursors of metastases. As used herein, the term CTC relates to a tumor cell in the peripheral blood or lymph and DTC relates to a tumor cell in the bone marrow.

CTCs and DTCs are extremely difficult to detect. They are exceptionally rare and may be difficult to distinguish from healthy cells. Existing approaches for detecting CTCs and DTCs have limitations in sensitivity and/or specificity, leading to many healthy cells being mischaracterized as cancerous, and many cancer cells being missed in the analysis.

The current established and used biomarkers for detection and enrichment of tumor cells from the blood or bone marrow of patients are strongly limited to epithelial properties and downregulated in mesenchymal tumor cells. Detection is done using the “epithelial cell adhesion molecule” (EpCAM) or other epithelial markers like cytokeratin (CK). Among a pool of epithelial CTCs there are tumor cells that have undergone or are undergoing “epithelial-to-mesenchymal transition” (EMT) and lost much of their epithelial phenotype (to allow them to detach from the primary tumor actively). Such CTCs and DTCs are thought to be major drivers in metastasis formation and express epithelial markers only in low levels, making their isolation or detection with these markers difficult. EpCAM is a transmembrane protein with an extracellular domain and is suitable both for the detection and the isolation of CTCs and DTCs expressing large enough levels of epithelial markers. In contrast, cytokeratins are cytoplasmic proteins and are only suitable for the detection of CTCs and DTCs, but not for the isolation of the cells.

Patent Application CN110234348A entitled “Anti-domain Protein Containing CUB 1 (CDCP1) Antibody, Antibody Drug Conjugate and its Application Method”, claiming priority to US201662435509, discloses a cancer detection and diagnosis method carried out on any biological sample such as biopsy, histotomy or body fluid (for example, urine, saliva, blood, tear, sperm or milk). Detection can involve western blot analysis, immunohistochemistry (IHC) analysis, immunofluorescence (IF) analysis, flow cytometry, FACS analysis, ELISA and immunoprecipitation. Alternatively, a biological sample comprising whole cells (for example, circulating tumor cells (CTCs)) can be used for immunohistochemistry (IHC), flow cytometry (FC) and immunofluorescence (IF). While this publication is concerned with detection of cancer, it does not mention that CDCP1 can be used in the isolation of CTCs. In particular, the methods that are described in the context of CTCs are immunohistochemistry (IHC) and immunofluorescence (IF), which are detection, but not isolation methods. The third mentioned application is flow cytometry (FC), which can be used for the detection and also for the isolation of the cells. However, for the isolation of the cells, the method FACS (fluorescence-activated cell sorting) is not mentioned, which would be mandatory for the intended application of cell isolation.

WO2013036620A1 entitled “Methods and Compositions for Cytometric Detection of Rare Target Cells in a Sample” discloses cytometric methods for the detection of rare target cells, such as circulating tumor cells (CTCs), in a biological sample such as blood. Aspects of the methods include contacting the sample with first and second binding members that specifically bind to a marker of the rare target cell, and cytometrically assaying the sample for the presence of cells comprising bound first and second binding members to detect the rare target cell in the sample. While this publication describes some general approaches for the detection of rare cells in a sample, it is limited to detection. Samples are analyzed for the presence of the cells. There is no mention of any further processing of the sample, including isolation. In addition, neither CDCP1 is mentioned here, nor the fact that for isolation of the cells at least one antibody has to bind to the extracellular domain of the target protein. In principle, two antibodies can also bind to a protein that is exclusively present in the cytoplasm. There is no attempt in this patent to differentiate between cytoplasmic (sole detection) and transmembrane (isolation and detection) proteins.

WO2019084319A1 discloses antibodies and antibody fragments that specifically bind the CUB domain-containing protein 1 (CDCP1) and their methods and uses in treating and detecting cancers. There is however only mention of solid tumors in this publication, with no mention of CTCs or DTCs, and thus no relation to isolation and detection of the cells.

U.S. patent application No. 2007/0031419 entitled “Methods and Compositions for Treating Diseases Targeting CDCP1” concerns a method for diagnosing or detecting a disease in a subject, the method comprising: determining a test level or test activity of CDCP1 protein in a disease cell from the subject, for example using an antibody that specifically binds to an antigenic region of CDCP1, and determining a control level or control activity in a cell from a healthy subject, wherein the disease is related to abnormal expression or function of CDCP1 protein, and wherein the test level or test activity in the cell from the subject is different from the control level or control activity in a cell from a healthy subject is indicative of the presence of the disease.

U.S. Pat. No. 10,874,728 “Tumor Vascular Marker-Targeted Vaccines” teaches inter alia a method of suppressing the growth of a tumor in a subject, wherein the vasculature supplying said tumor comprises a tumor vasculature marker (TVM). The method comprises the steps of: (a) identifying expression of said TVM by said tumor by contacting said subject with a labeled compound that binds said TVM or a nucleic acid molecule encoding said TVM; (b) detecting said label; (c) contacting said subject with an antibody to said TVM, wherein said antibody is labeled with a radionuclide to deliver cytotoxic radiation to tumor vasculature expressing said TVM; and (d) contacting said subject with said TVM or with a nucleic acid construct encoding said TVM to induce an immune response against said TVM. In one embodiment, the TVM is CDCP1, i.e., CDCP1 is used as a target for the treatment. In principle, an anti-CDCP1 antibody can bind to CDCP1, in which a cytotoxic compound is conjugated to the antibody so that CDCP1 positive cells can be eliminated by this cytotoxic reaction.

U.S. Pat. No. 7,741,114 discloses a method for analyzing a tissue biopsy sample, a bone marrow biopsy sample and/or a blood sample, comprising the steps of: contacting said sample with a monoclonal antibody, wherein the antibody binds to an epitope of CUB domain-containing protein 1 (CDCP1) which is the same as that bound by an antibody which is produced by the hybridoma cell lines CUB1, CUB2, CUB3 or CUB4, detecting binding of said cells to said antibody; and correlating binding of said cells to said antibody with the presence of CDCP1 antigen on said cells. The disclosure relates, in particular, to monoclonal antibodies, or fragments thereof, which are produced by the hybridoma cell lines CUB1, CUB2, CUB3 and CUB4. The inventors were surprisingly able to isolate the antibodies using the antigen CDCP1. However, no disease state of the patient from which the sample is taken is described. The publication describes the detection of CDCP1 positive cells in the blood in common with no relation to cancer or other diseases, so it might include the detection of CDCP1 in normal blood cells. The detection of CTC or DTC released from a solid tumor is not mentioned. Isolation is also not mentioned.

It is an object of the invention to provide a method particularly suited for the isolation and preferably isolation plus detection of cells with mesenchymal attributes, meaning cells showing only low level of keratin (like cells of the cell line MDA-MB-231) or almost negative for keratin like the DTC cell line from the bone marrow of a breast cancer patient BC-M1. CTCs or DTCs with mesenchymal or epithelial/mesenchymal phenotype are thought to be major drivers in metastasis formation. As the current established and used biomarkers (EpCAM, cytokeratins) for detection and enrichment of tumor cells from the blood of patients are strongly limited to epithelial properties and downregulated in mesenchymal tumor cells, the present invention aims to close this “detection gap” for tumor cells with mesenchymal attributes and to identify new cell surface biomarker proteins for such cells.

It is further an object of the invention to provide an improved method for isolation and preferably isolation and detection of CTCs and DTCs from blood, lymph or bone marrow. Isolation of CTCs and DTCs allows for a subsequent genetic or other analysis, e.g. for developing therapies. The biomarker proposed by the invention might function as potential immune target for personalized therapies. For example, highly personalized approaches, like the chimeric antigen receptor T-cell (CAR-T) technology, are an option to specifically attack cancer cells while reducing unwanted side effects. CARs usually contain specificity-conferring extracellular antibody single chain variable fragment (scFv), a CD3z-domain and intracellular costimulatory domains. The procedure begins with the isolation of T-cells from the blood of a patient, followed by a genetic modification to recognize the surface protein of interest and re-injection into the donor.

### SUMMARY OF THE INVENTION

The core of the invention is to use “CUB domain-containing protein 1”, abbreviated CDCP1, as a biomarker for the isolation and optionally isolation plus detection of CTCs and DTCs having a mesenchymal phenotype (mCTC, mDTC) or a hybrid epithelial/mesenchymal phenotype (emCTCs, emDTCs). It has been found that CDCP1 is, for example, strongly expressed in CTCs from breast cancer, in particular triple-negative breast cancer. The invention can, however, also be used in the context of other cancers with a high potential to form metastases, e.g. prostate cancer, pancreatic cancer, head and neck cancer, lung cancer or malignant mesothelioma.

CDCP1 is known as being relevant for cancer metastasis and as a marker for triple-negative breast cancer (see, for example, Turdo, F. et al. 2016, CDCP1 is a novel marker of the most aggressive human triple-negative breast cancers, Oncotarget 7: 69649-69665, doi:10.18632/oncotarget.11935). Antibodies against CDCP1 for the treatment and detection of cancer are also known (WO 2019/084319, US 2009/196873), and an ELISA detecting CDCP1 has also been described (Chen, Y. et al. 2017, Development of an enzyme-linked immunosorbent assay for detection of CDCP1 shed from the cell surface and present in colorectal cancer serum specimens, J Pharm Biomed Anal. 139:65-72, doi: 10.1016/j.jpba.2017.02.047). It has not been described or suggested to isolate, or to both isolate and detect, CTCs or DTCs using CDCP1.

Isolation can, for example, be done via magnetic-activated cell sorting (MACS®), a method for separation of various cell populations depending on their surface antigens (CD molecules), using anti-CDCP1 antibodies. The inventors developed a specific MACS-based system for isolating CTCs or DTCs expressing CDCP1. Isolation is, however, not limited to this method, and alternatively isolation can be done, for example, by a specially adapted commercial system called “CELLSEARCH” (see, for example, Coumans F, Terstappen L., 2015, Detection and Characterization of Circulating Tumor Cells by the CellSearch Approach, Methods Mol Biol. 1347:263-78, doi: 10.1007/978-1-4939-2990-0_18; Mesquita B, et al. 2017, Molecular analysis of single circulating tumour cells following long-term storage of clinical samples, Mol Oncol. 11(12):1687-1697, doi: 10.1002/1878-0261.12113) or by other methods, for example by immune cell isolation methods using immobilized anti-CDCP1 antibodies (e.g. anti-CDCP1 antibody coated matrices) or other binding molecules (e.g. aptamers) specifically binding to CDCP1. Methods using, for example, antibody-coated microstructures are also encompassed by the invention.

In the case of isolation plus detection the present invention requires the contacting of the cells with at least 2 antibodies, for example:

1. capture with an anti-CDCP1 antibody targeting the extracellular domain of CDCP1, followed by detection of the captured cells by a labeled secondary antibody targeting the bound anti-CDCP1 antibody, or

2. capture of the cells with a first anti-CDCP1 antibody targeting the extracellular domain of CDCP1, followed by the detection of the captured cells by a labeled secondary antibody targeting a second anti-CDCP1 antibody, bound to the intracellular domain of CDCP1. Alternatively, the second anti-CDCP1 antibody can itself be fluorescently labeled.

An anti-keratin antibody, i.e. an antibody directed against cytokeratin, may additionally be used for detecting cytokeratin-positive cells, thus to distinguish between CTCs and DTCs having a mesenchymal phenotype (mCTC, mDTC), which would not test cytokeratin-positive, and hybrid epithelial/mesenchymal phenotype (emCTCs, emDTCs), which would test cytokeratin-positive.

By way of comparison, U.S. Pat. No. 8,524,493 entitled “Released Cytokeratins as Markers for Epithelial Cells” teaches a protocol for the EPISPOT assay with the detection of the cells by released keratins. In this case, the cells are cultivated on a synthetic membrane. In contrast, MACS is performed in liquid solution, which allows subsequent handling of the cells. Moreover, the present invention is particularly suitable for the isolation and preferably additional detection of cells with mesenchymal attributes, meaning that these cells show only low level of keratin (MDA-MB-231) or are almost negative for keratin (BC-M1).

### BRIEF DESCRIPTION OF THE DRAWINGS

Further details and features of the invention will be described on the basis of the following figures, showing:

FIG. 1. Domain structure of CDCP1. a) Full-length membrane-bound CDCP1. b) Truncated version of membrane-bound CDCP1, cleaved within the extracellular domain and releasing a soluble fragment,

FIG. 2. Simplified representation of capture and detection of CDCP1 positive CTC by an immunomagnetic cell separation method, using two different approaches. In both approaches an anti-CDCP1 antibody (“capturing” antibody), which is directed to the extracellular domain of CDCP1, was coupled to magnetic nanoparticles. In the first approach (FIG. 2a) a labeled secondary antibody directed against the anti-CDCP1 antibody or against the complex composed of the anti-CDCP1 antibody, the magnetic nanoparticle and the components (e.g. biotin and streptavidin) coupling the magnetic nanoparticle and the anti-CDCP1 antibody, is used to detect CDCP1⁺ CTCs, in the second approach (FIG. 2b) a second primary anti-CDCP1 antibody, directed against the intracellular domain of CDCP1, and a secondary antibody directed against the second primary anti-CDCP1 antibody are used to detect CDCP1⁺ CTCs,

FIG. 3 Schematic illustration of an example of a cell surface molecule-dependent MACS isolation procedure. CTCs carrying the cell surface molecule (e.g. a transmembrane protein) are captured by an antibody directed against the cell surface molecule. The antibody is coupled to a magnetic nanoparticle, and the complex of antibody bound to the CTC and the magnetic nanoparticle can be retained in a column by a magnet (FIG. 3a) and subsequently released from the column by removing the magnet (3b),

FIG. 4. A: Detection of CDCP1 in CTC from a breast cancer patient by a first approach. B: Detection of CDCP1 in CTC from a breast cancer patient by a second approach, and

FIG. 5A: Western Blot analysis for CDCP1 of different breast cancer cell lines and DTC cell lines (BC-M1, LC-M1, PC-E1). B: Western Blot analysis for epithelial marker proteins. Keratin was detected by two different pan-keratin specific antibodies (A45/BB3, AE1/AE3). Alpha Tubulin served as a loading control. MDA-231: MDA-MB-231, MDA-468: MDA-MB-468. C: Detection of CDCP1 on DTC of a breast cancer patient.

### DETAILED DESCRIPTION OF THE INVENTION

As explained above, there is a specific pool of CTCs and DTCs that have undergone or are undergoing “epithelial-to-mesenchymal transition” (EMT) and lost much of their epithelial phenotype (to detach from the primary tumor actively). Such CTCs and DTCs express epithelial markers only in low levels, making it difficult for their detection/isolation with these markers.

“Epithelial-to-mesenchymal transition” EMT is a normal process in embryonic development, organogenesis, gastrulation and development of the peripheral nervous system which causes epithelia-mesenchymal plasticity and enables these cells to migrate and form different tissues and organs. The process of EMT plays a relevant role in wound healing and tissue regeneration. Cancer cells from the primary tumor also take advantage of this program to start invasion and migration. As a consequence, metastases occur which are primarily responsible for cancer-related deaths. Motility and invasiveness are essential requirements for metastatic spread of malignant cells.

EMT describes a process by which tumor cells can acquire mesenchymal attributes thereby gain attributes like increased migratory capacity. Alternatively, tumors may originate from cancer stem cells. In breast cancer, cancer stem cells are malignant transformed (adult) breast stem cells. Cancer stem cells retain limitless replication potential and high migratory capacity rendering ideal candidates for the formation of distant metastasis. In addition, cancer stem cells are able to undergo uneven cell division. That means, after cell division one daughter cell is again a cancer stem cell and the other daughter cell can begin cellular differentiation. During the differentiation, the descendants gradually increase epithelial attributes, with the upregulation of epithelial marker proteins like keratins. Cancer stem cells are tumor cells with strong manifestation of a mesenchymal phenotype and very low expression of epithelial proteins like EpCam or keratins. Interestingly, the protein expression profile of cancer stem cells and EMT passed cells is very similar (see below).

Before detaching from primary tumor, the number of cell-cell contacts of the tumor cells decrease by downregulation of proteins responsible for adherence which in turn results in the loss of the apical-basal polarity. After numerous genetic changes the tumor cells are released from the primary tumor, penetrate the basement membrane and intravasate actively or passively the blood circulation or the lymphatic vessels. The CTCs are exposed to physical stress like shear forces in the blood stream or collision with blood cells. Additionally, they have to avoid anoikis, a process which normally leads to apoptosis by the lack of cell-ECM contacts and escape the immune system. After reaching a distant organ, tumor cells extravasate the blood circulation by binding to the endothelium after reaching small capillaries. After extravasation, DTCs die, remain in tumor dormancy or resume proliferation to found the outgrowth of macrometastasis. In breast cancer the outgrowth of metastasis can take several years after removal of the primary tumor. One reason for the late metastatic relapse is the dormancy state of DTCs with low proliferation rates resulting in weak therapy effects in this period followed by resuming signal-triggered proliferation. CTCs and DTCs with mesenchymal attributes or after passing through EMT have advantages in motility but not in proliferation. In general, after complete EMT, the expression of epithelial proteins like EpCAM or E-Cadherin is downregulated due to the loss of cell-cell connections, cytoskeletal alterations and changes in keratin expression patterns followed by the final upregulation of mesenchymal proteins like Vimentin and N-Cadherin.

The present invention thus uses the CUB domain-containing protein 1 (CDCP1) as a biomarker for the isolation, preferably the isolation and detection of CTCs or DTCs having a mesenchymal phenotype (mCTC, mDTC) or a hybrid epithelial/mesenchymal phenotype (emCTC, emDTC). It has been found that CDCP1 is, for example, strongly expressed in CTCs from breast cancer, in particular triple-negative breast cancer. The invention can, however, also be used in the context of other cancers with a high potential to form metastases, e.g. prostate cancer, pancreatic cancer, head and neck cancer, lung cancer or malignant mesothelioma.

The term “isolation” in relation to targeted cells like CTCs or DTCs refers to the separation of these cells from a biological sample containing the cells, for example, from peripheral blood, lymph, bone marrow or the like. The term includes the separation of these cells from other, not targeted cells, e.g. the separation of CDCP1-positive CTCs or DTCs from the biological sample medium and other cells in the same sample, e.g. CDCP1-negative CTCs or DTCs. The term encompasses enrichment of the targeted cells, i.e. increasing the concentration of the targeted cells by removal of fractions not containing targeted cells, or removal of targeted cells to a collection medium, or by accumulating targeted cells in a part or compartment of a container or in an area of an object slide.

The term “detection” in relation to targeted cells like CTCs or DTCs refers to identification of the targeted cells in a biological sample, in a medium enriched with the targeted cells or in a medium at least mainly, preferably exclusively containing the targeted cells. Identification means suitably labeling the cells and qualitatively or quantitatively detecting the presence of the cells.

The term “subject” as used herein refers to a non-human animal, preferably a non-human mammal, for example a cat, dog, horse, cattle, or to a human.

The term “nanoparticle” as used herein relates to particles of a size of 1 to ≤100 nm. In relation to a plurality of nanoparticles the term means that the particle size of at least half of the particles in the number size distribution of the plurality of nanoparticles is 100 nm or below.

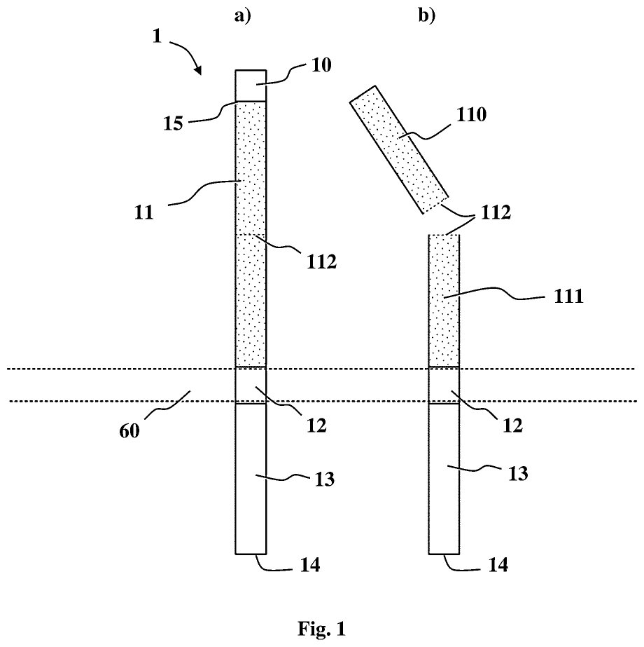

CDCP1 is a single-pass type I membrane glycoprotein with an intracellular, a membrane and an extracellular domain (FIG. 1). For a sequence of CDCP1 see, for example, UniProtKB Q9H5V8 (sequence version 3 of Jan. 11, 2011, entry version 147 of Jun. 2, 2021). The extracellular domain consists of three CUB (C1r/C1s, Uegf, Bmp1) domains and the complete sequence contains 14 sites for N-glycosylation as well as 20 cysteine residues potentially involved in formation of disulfide bonds. Structural features comprise a full sequence length of 836 amino acids consisting of a 29 amino acid signal peptide 10, a cytoplasmic (intracellular) domain 13 of 150 amino acids, a transmembrane domain 12 of 21 amino acids and an extracellular domain 11 of 636 amino acids. The calculated molecular weight of this protein is around 90.1 kDa, however the observed molecular weight in Western Blot analysis is around 135 kDa due to N-Glycosylations.

In FIG. 1, the structure of CDCP1 (denoted with reference numeral 1) is schematically depicted. CDCP1 1 is anchored in the cell membrane 60 via a transmembrane domain 12 (amino acids 666-686). The N-terminal 15 is located extracellularly, the C-terminal 14 is located at the end of the intracellular (cytoplasmic) domain 13. The extracellular domain 11 has a serine protease cleavage site 112, such that the extracellular domain 11 can be split into a truncated membrane-bound extracellular domain fragment 111 and a soluble CDCP1-fragment 110 (glycosylated) of 65 Da (see FIG. 1b). The extracellular domain can, for example, be a target for MACS. Both the extracellular domain 11 and the cytoplasmic domain 13 can be a target for immunofluorescence detection, for example. The soluble CDCP1 fragment 110 can be a target for an ELISA.

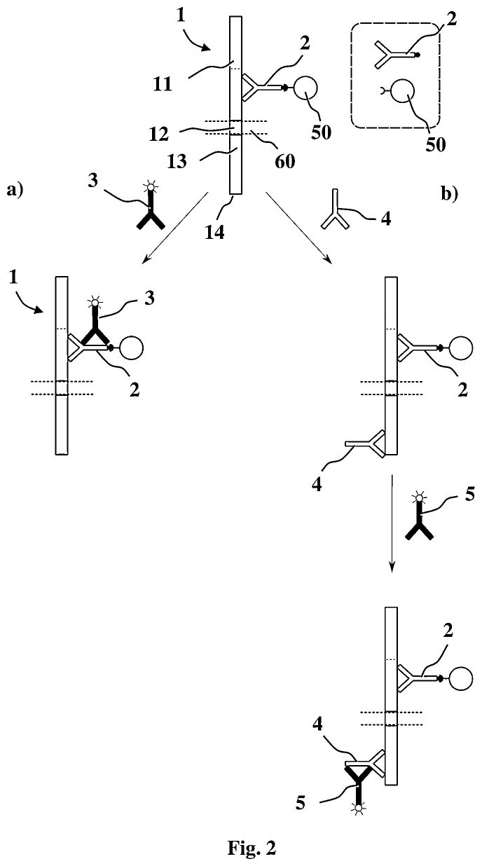

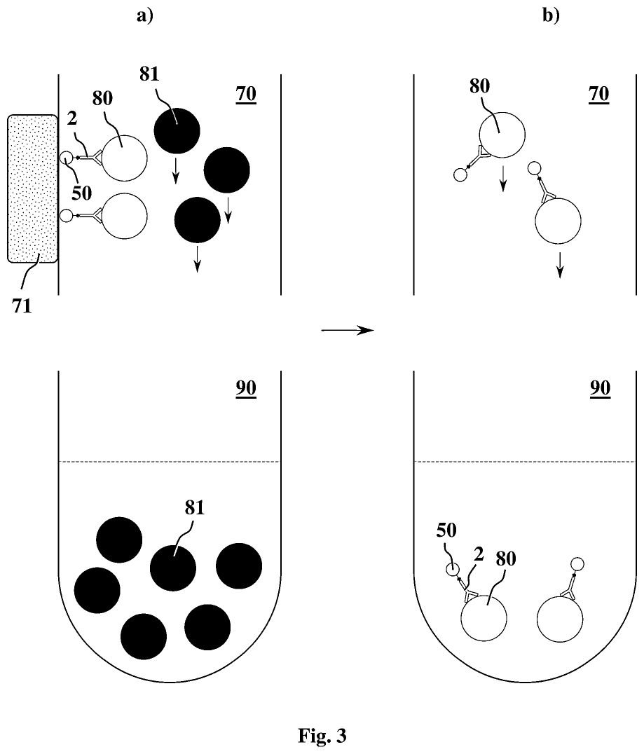

As one example, the method of the invention can be carried out using magnetic-activated cell sorting (MACS®), using magnetic-activated anti-CDCP1 antibodies, i.e. anti-CDCP1 antibodies coupled to magnetic nanoparticles, as described below in more detail in relation to FIGS. 2, 3. However, isolation of CTC and/or DTC can be achieved via an anti-CDCP1 antibody coupled to any suitable substrate or functionalized on any surface.

FIG. 2 shows an example of an embodiment of the method of the invention using magnetic activated cell sorting (MACS®) for the isolation of CDCP1⁺ cells, i.e. CDCP1-positive cells. CDCP1⁺ cells are here represented by CDCP1 (1) anchored in the cell-membrane (60). In a first step, a CTC or DTC carrying CDCP1 on its cell-surface, is captured with a biotinylated first primary antibody (2) directed to the extracellular domain (11) of CDCP1 (1), the first primary antibody (2) being coupled to a magnetic nanoparticle (50) via streptavidin linked to the magnetic nanoparticle (50). The first primary antibody (2) is preferably a polyclonal antibody, particularly preferred a (polyclonal) antibody binding to the part of the extracellular domain (11) of CDCP1 that is not cleaved off, i.e. binding also to the truncated version of CDCP1. CDCP1⁺ CTC/DTCs can be isolated via MACS®, as described in relation to FIG. 3. It should, however, be noted, that the isolation can be performed using a modified CellSearch system (with CDCP1 instead of EpCAM as a marker) or other isolation procedures, e.g. via anti-CDCP1 antibodies bound to a solid phase other than a magnetic nanoparticle (50). For the subsequent detection of CTC/DTCs, two approaches are shown. In a first approach (see FIG. 2a) a fluorescently labeled first secondary antibody (3) directed against the first primary anti CDCP1-antibody (2), or against the complex composed of the first primary anti-CDCP1-antibody (2), the magnetic nanoparticle (50) and the components coupling the magnetic nanoparticle (50) to the first primary anti-CDCP1-antibody (2) (biotin and streptavidin in this case), is added in order to detect CDCP1-positive CTC/DTCs. In another approach (see FIG. 2b), a second primary anti-CDCP1-antibody (4), directed against a preferably C-terminal region of the intracellular (cytoplasmic) domain (13) of CDCP1 (1), is added to bound to CDCP1 (1), and a fluorescently labeled second secondary antibody (5) directed against the second primary anti CDCP1-antibody (4) is added in order to detect CDCP1-positive CTC/DTCs.

The method of the invention can be adapted to various geometries and topologies. First, the coupling of an anti-CDCP1 antibody is not limited to coupling to magnetic nanoparticles, as described above for FIG. 2. Rather, an anti-CDCP1 antibody can be coupled by different methods to any suitable material, e.g. any solid phase material used in the prior art for immobilizing antibodies. Such coupling methods may be for example the application of reactive cosslinkers like NHS esters (N-hydroxysuccinimide esters) and imidoesters, which allow the coupling of anti-CDCP1 antibodies to a suitable material or surface. Another option is the covalent conjugation of the anti-CDCP1-antibody to fluorescent (non-magnetic or magnetic) nanoparticles. This approach could be used for detection (fluorescent nanoparticles) or isolation/enrichment (magnetic nanoparticles). Coupling of an anti-CDCP1 antibody to a fluorescent magnetic nanoparticle is advantageous in that this direct approach would work without a secondary antibody reaction. As already mentioned, different materials/surfaces instead of magnetic nanoparticles to which the anti-CDCP1 antibody is coupled can be applied. Such material may be for example Sepharose® crosslinked, beaded-form of agarose, or nanoparticles (fluorescence labeled or unlabeled), but other materials or surfaces may also be applied. For example, the cell collector (see US patent US10022109B2) can be adjusted to the capture of CDCP1-positive CTCs or DTCs by functionalizing the device with anti-CDCP1 antibodies. Another example is the detection/isolation by a modified commercial system called “CellSearch”, which is an immunomagnetic technology that uses anti-EpCAM antibodies coupled to ferrofluid nanoparticles for separation of EpCAM-expressing cells. Supplement or substitution of anti-EpCAM antibodies with anti-CDCP1 antibodies coupled to ferrofluid nanoparticles in the CellSearch approach enriches CDCP1-positive CTC or improves the number of enriched CTC, respectively.

Currently, immunomagnetic methods (MACS) are widely-used to isolate tumor cells. For example, the immunomagnetic isolation via the extracellular domain of EpCAM is used to capture EpCAM-positive CTCs and DTCs in metastatic breast cancer. This approach enables the isolation of CTCs or DTCs with an epithelial phenotype. The disadvantage of this approach is the loss of EpCAM-negative CTCs and DTCs. EpCAM-negative tumor cells might have undergone a complete EMT which leads to a mesenchymal phenotype or a partial EMT which leads to a hybrid epithelial/mesenchymal phenotype. Mesenchymal CTCs are thought to be more invasive than the epithelial counterparts but the hybrid epithelial/mesenchymal phenotype is linked with metastasis formation and metastatic relapse. These findings indicate that there is need for a cell surface molecule which can be used for isolating CTCs or DTCs, for example a MACS-based method for the isolation of CTCs with mesenchymal or hybrid epithelial/mesenchymal attributes. The inventors have found that the large extracellular domain of CDCP1 is applicable for, for example, MACS-based isolation of CTCs/DTCs with mesenchymal or hybrid epithelial/mesenchymal phenotype. Due to the fact that different biological variants of CDCP1 correlate with dissemination and invasion of tumor cells, CDCP1-positive tumor cells might provide information about metastasis formation-potential and metastasis development.

For the labeling procedure an indirect and a direct variant can be distinguished. The antigen of interest on the cell surface can be hybridized with an antibody or a biotinylated antibody followed by the binding of the super-paramagnetic nanoparticles (MACS-nanobeads, size range: 20-100 nm) which are conjugated to a secondary antibody (recognizing the primary antibody) or to Streptavidin (recognizing the biotinylated antibody). This approach represents the indirect technique. The faster direct approach is represented by the direct binding of MACS bead-conjugated antibodies to the cell surface antigen of interest. For the isolation procedure a positive and a negative selection can be differentiated. The positive selection enriches the cells harboring the antigen of interest on their cell surface and the cells lacking the cell surface antigen are discarded. For this purpose, the antigen-positive cells are captured with an antibody or other binding agent coupled to a magnetic nanoparticle and retained by a magnetic field. On the other hand, the negative selection isolates all cells lacking the cell surface antigen of interest by capturing them with a binding agent, e.g. antibody, linked to a magnetic nanoparticle, while the cells expressing the cell surface antigen are not captured and released from, for example, a MACS column. After successful labeling with magnetic nanoparticles the cells are applied to a ferromagnetic iron-column positioned in a magnetic field. In FIG. 3, the procedure is schematically shown for the positive selection of cells by a cell surface molecule-dependent MACS isolation procedure. Cells (80) are labeled with a biotinylated antibody (2) coupled via streptavidin to a magnetic nanoparticle (50). The antibody (2) is directed against a membrane-bound antigen, e.g. a membrane-bound protein having an extracellular domain, like CDCSP1. Nanoparticle-labeled cells (80) retain in the column (70) by the application of a magnetic field of a magnet (71) and unlabeled or antigen-negative cells (81) pass the column (70) and will be discarded, or collected in a collecting vessel (90). Thereafter (see FIG. 3b), the magnetic column (70) is removed from the magnet (71), the labeled cells (80) are eluted and collected in a collecting vessel (90), and further analysis can be performed. The complex composed of the biotinylated antibody (2) and the Streptavidin-conjugated magnetic nanoparticle (50) recognizes and binds to the target cell (80). The cell-antibody-nanoparticle complex binds to the MACS column (70) placed into a permanent magnet (71). The unlabeled cells (81) or cells negative for the surface protein are discarded after passing the column (70) without binding. After removal of the column (70) from the magnet (71) the labeled cells (80) are collected and represent an enriched population.

In principle, it is possible to carry out the isolation of CTCs or DTCs with only the anti-CDCP1 antibody coupled to the nanoparticles (or similar suitable substrates). The identity of the isolated cells can be confirmed, for example, by immunofluorescence by applying a second anti-CDCP1 antibody and a fluorescently labeled secondary antibody directed against the second anti-CDCP1 antibody, or a fluorescently labeled secondary antibody directed against the first CDCP1-antibody (anti-catching antibody). Additionally, an anti-keratin antibody may be used in combination with the second anti-CDCP1 antibody or the anti-catching antibody. Alternatively, the cells can be isolated and transferred to another vessel for subsequent procedures. Such procedures may include a cell lysis step followed by whole genome amplification followed by DNA sequencing of the genome to identify tumor-relevant gene mutations. Such gene mutations might be responsible for therapy resistance if a gene of a therapeutic target is mutated (e. g. EGFR). An alternative evolving approach is the analysis of the isolated cells on proteome level, for example by single cell mass spectrometry. In that case cancer relevant target structures (most therapy targets are proteins) can be directly assessed, which helps the prediction of therapy success of the patients. Depending on the isolation procedure, isolated cells are viable and thus can be used for subsequent functional in vitro (e.g., migration and invasion assay) and in vivo (e.g., patient-derived xenograft in zebrafish embryos) assays possibly after expansion in long-term cultures.

As an alternative to magnetic beads (nanoparticles), the anti-CDCP1 antibody can be coupled to an organic matrix such as Sepharose®, and this can be added to the sample. Sepharose forms a kind of slurry. When you have the complex CTC-anti-CDCP1-antibody-Sepharose in the sample, you can simply let the complex settle down (no centrifugation step necessary) and discard the supernatant. If you want to remove normal cells that got stuck in the slurry, simply dissolve the slurry in a suitable buffer and let it settle down again and discard the supernatant. Accordingly, the invention can be carried out with an organic matrix (e. g. Sepharose) having anti-CDCP1 antibodies coupled via reactive functional groups, wherein the separation involves gravitation force, for example by settlement of the CTC-anti-CDCP1-antibody-Sepharose complex or centrifugation.

Material and Methods

Magnetic-activated cell sorting (MACS). For the formation of an antibody-bead-complex 0.4 μg (2 μl) biotinylated polyclonal goat anti-human CDCP1 (catalogue number: BAF2666, Novus Biologicals, Centennial, USA) and 15 Streptavidin MicroBeads (diameter: 50 nm) were incubated with 100 μl MACS Buffer (composed of MACS BSA Stock Solution and autoMACS® Rinsing Solution (1:20) (both Miltenyi Biotec, Bergisch Gladbach, Germany) for 30 minutes at 22° C. and shaking at 600 rpm. In parallel, the isolation of mononuclear cells and tumor cells from peripheral blood was performed. Therefore, whole blood sample from a patient with metastatic breast cancer (6.5 ml) was diluted with PBS to a volume of 15 ml. Diluted blood was gently layered on top of 20 ml of Ficoll Paque density gradient medium (GE Healthcare, Chicago, USA). After centrifugation for 30 minutes at 400×g with soft acceleration and braking, the upper layer containing plasma was discarded and the mononuclear cell layer at the interface was transferred to a new centrifuge tube. Diluted with PBS to a volume of 40 ml, mononuclear cells and tumor cells were pelletized by centrifugation at 400×g for 10 minutes.

The cell pellet was diluted in 800 μl MACS Buffer and divided into two equal parts. Each part was added to a prepared antibody-bead-complex and incubated for 60 minutes at 22° C. and shaking at 600 rpm. Cells were washed with 1.4 ml MACS Buffer and centrifuged at 300×g for 10 minutes. The supernatant was discarded and the cell pellet was resuspended in 1.5 ml MACS Buffer. The cell suspension was loaded on a MS-column (Miltenyi Biotec, Bergisch Gladbach, Germany), after the column was placed on an OctoMACS™ Separator (Miltenyi Biotec, Bergisch Gladbach, Germany) and prepared by rinsing with 1.5 ml of MACS Buffer. The cells were washed three times with 500 μl MACS Buffer before the column was removed from the magnet for elution of the cells. The elution was performed directly on a microscope slide with big cytospin funnel by adding 2 ml MACS Buffer (1.5 mL by gravity flow and 0.5 ml by using a plunger) to the column. The microscope slides were dried overnight and analyzed by immunofluorescent staining.

Immunofluorescent staining of the microscope slides. To prevent detaching of cells the cell-containing area on the microscope slide was surrounded with the DAKO-pen (Dako/Agilent, Santa Clara, USA). After each described step, except between blocking step and antibody incubation step, cytospins were washed with PBS 3 times for 2 minutes each. All antibodies were diluted in 10% human AB serum (blocking solution). First, cells were fixed by incubation with 2% PFA for 10 minutes. Blocking was carried out with blocking solution for 30 minutes. One of the obtained cytospins was incubated with a primary antibody against the C-terminus of CDCP1 (Cell Signaling Technology, Danvers, USA) at a dilution of 1:100 in 10% AB-serum in DPBS for 60 minutes at room temperature, followed by incubation with Alexa 546 goat anti rabbit IgG antibody at a dilution of 1:200 in 10% AB-serum in DPBS for 45 minutes, to detect the full-length CDCP1 protein. The other cytospin was incubated with Alexa546 donkey anti-goat secondary antibody at a dilution of 1:200 in 10% AB-serum in DPBS for 45 minutes at room temperature to detect the biotinylated capturing CDCP1 antibody. For the detection of cytokeratins (tumor cell marker), CD45 (leukocyte marker) and DAPI, both cytospins were then incubated with an antibody cocktail (Alexa488-conjugated anti-Cytokeratin (AE1/AE3) antibody at a dilution of 1:300, APC-conjugated anti-CD45 antibody at a dilution of 1:200 and DAPI at a dilution of 1:1,000 in 10% AB-serum in DPBS) for 60 minutes at room temperature. After washing, cells were covered with one drop of mounting medium Prolong™ Gold Antifade (Thermo Fisher Scientific, Waltham, USA) and the cover slip was fixed with fixogum (Marabu, Ludwigsburg, Germany). The cytospins were analyzed manually using the microscope Axio Observer with Axiocam 702 mono (Carl Zeiss, Oberkochen, Germany) with the software ZEN 3.0 (blue edition) (Carl Zeiss, Oberkochen, Germany).

Results

An overview about the experimental approach for the isolation of circulating tumor cells (CTCs) from the blood of the breast cancer patient is shown in FIG. 1-3, which show

FIG. 1: Domain structure of CDCP1 (1). The full-length mature protein consists of an extracellular domain (11) with glycosylation, a transmembrane domain (12) and a cytoplasmic domain (13). In the extracellular domain (11), CDCP1 can be cleaved by a serine protease resulting in a soluble fragment (110) and a membrane-bound variant of CDCP1 (1) having a truncated extracellular domain (111). The soluble 65 kDa-fragment (after cleavage) could be used for the detection in the blood of cancer patients by ELISA as a non-invasive diagnosis method. The full-length and the membrane-bound variant could be analyzed by immunofluorescence (IF) using an anti-CDCP1 antibody (2) directed against this domain. For CTC capture by MACS, the extracellular domain (11) was used. The cytoplasmic domain (13) is suitable for detection by immunofluorescence (IF).

FIGS. 2, 3: Capture and detection of CDCP1 positive CTC by MACS by two different approaches. In both approaches an anti-CDCP1 antibody (2), which is directed to the extracellular domain (11) of CDCP1 (first primary antibody (2)), was coupled to magnetic nanoparticles (50). This antibody (2) was raised in goat. After CTC capture and purification, a complex composed of the CDCP1-positive CTC, the anti-CDCP1 antibody (2) and the magnetic nanoparticle (50) is present. In approach 1 (FIG. 2a), a fluorescently labeled first secondary antibody (3) against goat was used. This first secondary antibody (3) binds to the complex of the first primary antibody (2) and the magnetic nanoparticle (50) to confirm that this complex indeed bound to CTC. In approach 2 (FIG. 2a), the complex consisting of CDCP1-positive CTC, first primary antibody (2) and magnetic nanoparticle (50) was analyzed using a second primary anti-CDCP1 antibody (4). The epitope targeted by the second primary anti-CDCP1 antibody (4) is in the C-terminal region of CDCP1, i.e. in the cytoplasmic domain. The second primary anti-CDCP1 antibody (4) was raised in rabbit. The second primary anti-CDCP1 antibody (4) was then detected using a labeled second secondary antibody (5), in this case an anti-rabbit specific secondary antibody containing a fluorophore. This approach provides additional evidence for the presence of CDCP1 in the CTCs as well as the presence of the C-terminal region of CDCP1.

Keratin staining of CTCs additionally performed in both approaches. In brief, CTCs were isolated by a first anti-CDCP1 antibody (2) directed against the extracellular domain (11) of CDCP1 (1) that was raised in goat. This antibody was conjugated with magnetic nanoparticles 50 allowing the isolation of CDCP1-positive cells by MACS (FIG. 3). The detection of CDCP1-positive cells in the eluate was performed by two different approaches on two different microscope slides (FIG. 2a, b). In approach 1 (FIG. 2a), the complex of anti-CDCP1 primary antibody (2) and magnetic nanoparticle (50) was detected by a first secondary antibody (3) that binds to the first anti-CDCP1 primary antibody (2) or the complex formed by the anti-CDCP1 primary antibody (2) and the magnetic nanoparticle (50) coupled by streptavidin/biotin. This approach confirms that the isolated CTCs in the eluate were positive for the complex of first anti-CDCP1 primary antibody (2) and magnetic nanoparticle (50), thus specifically caught by CDCP1 and the magnetic nanoparticles (50). In approach 2 (FIG. 2b), a second primary anti-CDCP1 antibody (4) specific to the C-terminal region of CDCP1 was applied. This approach allows the confirmation of CDCP1 in the isolated cells by a second primary anti-CDCP1 antibody (4) visualized by the 2nd secondary antibody (5). Moreover, this approach confirms that CTC with CDCP1 containing the cytoplasmic and the extracellular domain were isolated.

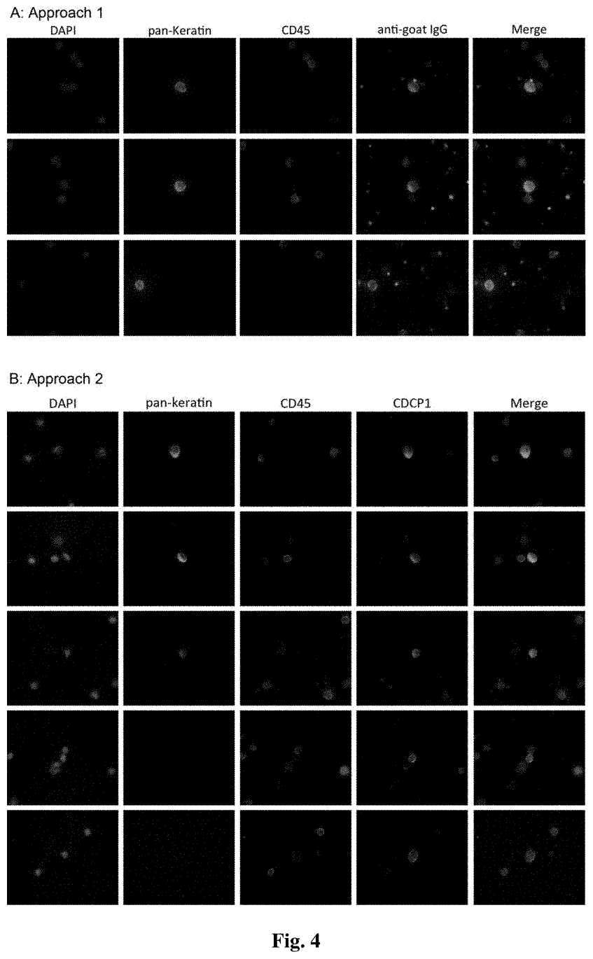

Cells that were detected by these approaches are shown in FIG. 4. CTC were detected by keratin staining and normal blood cells were detected by CD45 staining. CTC that were detected by approach 1 are shown in FIG. 4A. The figure shows:

A: Confirmation of CDCP1 in CTC from a breast cancer patient by approach 1. CTCs were subjected to keratin staining and normal blood cells were confirmed by CD45. CDCP1 was confirmed by an anti-goat IgG secondary antibody.

B: Confirmation of CDCP1 in CTCs from a breast cancer patient by approach 2. CTCs were subjected to keratin staining and normal blood cells were confirmed by CD45. CDCP1 was confirmed by an anti-CDCP1 antibody directed against the C-terminal domain of CDCP1. The two bottom panels show two CDCP1 positive cells that were CD45 and keratin negative. The merged images show an overlay of the DAPI, keratin, CD45 and the CDCP1 signals.

CDCP1 positive CTCs were negative for CD45 and positive for keratin as well as positive for the anti-goat IgG that binds against the anti-CDCP1 primary antibody. The small speckles in the anti-goat IgG channel are probably precipitated anti-CDCP1 primary antibody-magnetic bead complexes.

Keratin staining served as a confirmation that an isolated cell is a CTC (with some epithelial attributes) and to divide the isolated CDCP1 positive cells into normal CTC (keratin positive) and mesenchymal CTC.

CTC that were detected by approach 2 are shown in FIG. 4B. Three keratin positive CTC that were positive for CDCP1 and negative for CD45 are shown. CDCP1 was detected by the second primary anti-CDCP1 antibody 4 specific to the C-terminal region of CDCP1 (raised in rabbit). The precipitated magnetic nanoparticle complexes are not visible in this approach, because the anti-CDCP1 antibody coupled to these nanoparticles was raised in goat. In addition, two cells are shown that are negative for keratin and CD45, but are positive for CDCP1.

Accordingly, the inventors have successfully isolated and detected CTCs from the blood of a breast cancer patient by CDCP1 using MACS. Even though results from only one breast cancer patients is shown above, the procedure has been repeated to confirm detection of CTCs by two different detection approaches. Approach 1 shows that the isolated CTC were coupled to the magnetic nanoparticles confirming that these cells were specifically isolated by the magnetic nanoparticles and not due to unspecific carry-over from the blood sample. In approach 2, the presence of CDCP1 was confirmed by two different anti-CDCP1 specific antibodies. For CTC catching by MACS a first primary anti-CDCP1 antibody (2) directed against the extracellular domain (11) was used and for CDCP1 detection by immunofluorescence a second primary anti-CDCP1 specific antibody (4) directed against the cytoplasmic C-terminal tail of CDCP1 was used.

Detection of keratin positive CTC confirms that the method of the invention is suitable for the specific isolation of CTC by CDCP1. In addition, the method of the invention is suitable for the isolation of CTCs that are negative for keratin, thus CTC with mesenchymal attributes. In FIG. 4B two keratin negative cells that were positive for CDCP1 are shown. It is reasonable that such cells are CTC with mesenchymal attributes. The invention provides a tool for the detection, isolation and molecular analysis of CTC with mesenchymal attributes, which are considered as a probable candidate for the actual metastasis founding cells in breast cancer.

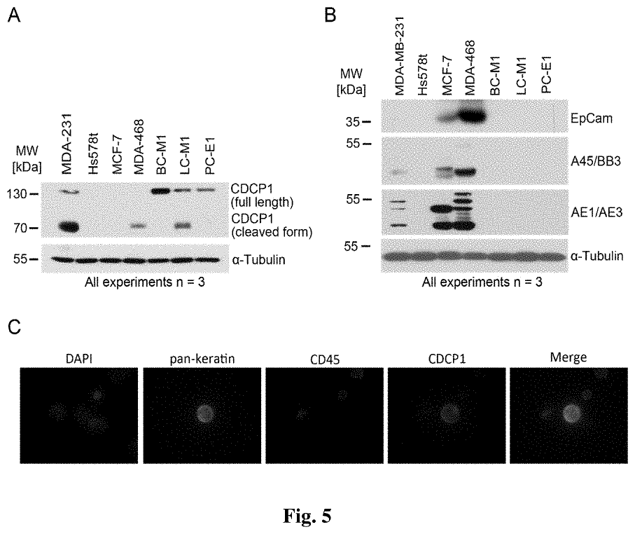

Further, the inventors performed Western Blot analysis for CDCP1 of cancer cell lines from different tumor entities (see FIG. 5). The full-length CDCP1 protein was detected in pancreatic cancer cell lines (Panc-1, MiaPaca2, BxPC3, PaCa5061), prostate cancer cell lines (PC-3, LNCaP, Du145), head and neck cancer (SAS, UM-SCC-5, UT-SCC 42a), lung cancer (H1299) and malignant mesothelioma (H2452, JL1) cell lines. Due to this widespread distribution of CDCP1 in different cancer entities the inventors conclude that CDCP1 can be applied for the detection and isolation of CTCs and DTCs for any epithelial cancer.

After confirmation of CDCP1 in CTC of breast cancer patients, the inventors investigated DTC cell lines that were generated from the bone marrow of breast cancer patients (FIG. 4). BC-M1 was from a breast cancer patient, LC-M1 was from a lung cancer patient, and PC-E1 was from a prostate cancer patient (FIG. 4). The full-length CDCP1 protein that is the target for the MACS approach was detected in the breast cancer cell line MDA-MB-231. High levels of the full-length CDCP1 protein was also detected in all three analyzed DTC cell lines from breast, lung and prostate cancer patients (FIG. 4A). Notably, these cell lines showed no detectable signals for the epithelial marker proteins of the keratin family and EpCam (FIG. 4B). EpCam is most commonly used for the isolation of tumor cells from liquid samples by magnetic beads. Therefore, the analyzed DTC populations represented by the cell lines BC-M1, LC-M1, and PC-El would remain undetectable by an anti-EpCam based MACS approach. In contrast, BC-M1, LC-M1, and PC-E1 are suitable target cells for an anti-CDCP1 based MACS approach. In accordance with the results shown for DTC cell lines, expression of CDCP1 on DTC was proven by immunofluorescence analysis of a bone marrow sample of a patient with breast cancer (FIG. 4C). In total, six CDCP1 positive DTC were detected. Therefore, the invention could be used to enrich CDCP1-positive DTC from bone marrow samples.

### Reference Signs

1 CDCP1

10 CDCP1 signal peptide (aa 1-29)

11 CDCP1 extracellular domain (aa 30-665)

110 CDCP1 extracellular domain soluble fragment

111 CDCP1 membrane bound truncated extracellular domain

112 CDCP1 extracellular domain cleavage site

12 CDCP1 transmembrane domain (aa 666-686)

13 CDCP1 intracellular (cytoplasmic) domain (aa 687-686)

14 CDCP1 C-terminal

15 CDCP1 N-terminal

2 1^{st} primary antibody (against CDCP1 extracellular domain; biotinylated); capturing antibody

3 1^{st} secondary antibody (against 1^{st} primary antibody)

4 2^{nd} primary antibody (against CDCP1 cytoplasmic domain)

5 2^{nd} secondary antibody (against 2^{nd} primary antibody)

50 magnetic nanoparticle (with bound streptavidin)

60 cell membrane (of CTC/DTC)

70 MACS column

71 Magnet

80 CTC/DTC (CDCP1⁺)

81 CTC/DTC (CDCP1⁻)

90 collecting vessel

Now that the invention has been described, I claim:

1. A method for isolation of circulating tumor cells (CTCs) and/or disseminated tumor cells (DTCs) with mesenchymal properties from a blood, lymph or bone marrow sample of a subject, comprising:
a. obtaining from the subject a sample containing cells to be isolated,
b. exposing the cells to anti-CDCP1 antibodies conjugated to particles or a matrix having a separation functionality for a time sufficient for CTCs and/or DTCs to attach to the anti-CDCP1 antibody conjugated particles or matrix,
c. separating the particles or matrix from the sample using the separation functionality of the particles or matrix, thereby isolating CTCs and/or DTCs with mesenchymal properties.

2. The method of claim 1, wherein the particles are magnetic nanoparticles, and wherein separating involves a magnetic field.

3. The method of claim 2, wherein the anti-CDCP1 antibodies conjugated to the magnetic nanoparticles are labelled with or contain at least one fluorophor.

4. The method of claim 1, wherein anti-CDCP1 antibodies are against full length CDCP1.

5. The method of claim 1, wherein anti-CDCP1 antibodies are against truncated length CDCP1.

6. The method of claim 1, wherein the matrix is an organic matrix having anti-CDCP1 antibodies coupled via reactive functional groups, and wherein separation involves sedimentation by gravity or centrifuge.

7. The method of claim 6, wherein the organic matrix is agarose.

8. The method as in claim 1, wherein the CTCs or DTCs having a mesenchymal phenotype (mCTC, mDTC) have a hybrid epithelial/mesenchymal phenotype (emCTC, emDTC).

9. A method for the isolation and detection of circulating tumor cells (CTCs) and/or disseminated tumor cells (DTCs) with mesenchymal properties from a blood, lymph or bone marrow sample of a subject, comprising:
a. obtaining a blood, lymph or bone marrow sample from the subject,
b. exposing the cells to a first anti-CDCP1 antibody,
c. isolating any bound CDCP1 positive cells from the sample,
d. exposing bound CDCP1 positive cells to a second antibody for detection of bound CDCP1 positive cells.

10. The method according to claim 9, wherein the second antibody is a labeled secondary antibody directed against the first anti-CDCP1 antibody, a second anti-CDCP1 antibody, or a labeled second anti-CDCP1 antibody, or a labeled secondary antibody directed against a second bound anti-CDCP1 antibody.

11. The method according to claim 9, further comprising isolation of T-cells from the blood of the subject, genetic modification of the T-cells to recognize CDCP1 surface protein and re-injection of the genetic modified T-cells into the subject.

12. The method according to claim 9, wherein isolation is done immunomagnetically using anti-CDCP1 antibodies coupled directly or indirectly to magnetic nanoparticles.

13. The method according to claim 12, wherein cells labeled with anti-CDCP1 antibodies coupled to magnetic nanoparticles are applied to a ferromagnetic iron-column positioned in a magnetic field, wherein labeled cells are retained in the column and unlabeled or antigen-negative cells pass the column and are discarded, and wherein the ferromagnetic iron-column is removed from the magnetic field and the labeled cells are eluted and available for further analysis.

14. A method for the isolation and detection of circulating tumor cells (CTCs) and/or disseminated tumor cells (DTCs) with mesenchymal properties from a blood, lymph or bone marrow sample of a subject, comprising:
a. obtaining a sample containing cells to be tested,
b. exposing the cells to a first anti-CDCP1 antibody for capturing of the cells,
c. isolating any bound CDCP1 positive cells from the sample,
d. exposing bound CDCP1 positive cells to a second anti-CDCP1 antibody and/or an anti-keratin antibody for the immunofluorescent detection of bound CDCP1 positive cells, wherein the anti-CDCP1 antibody is labelled with a different fluorophor from the anti-keratin antibody,
e. classifying CDCP1/keratin double positive isolated cells as emCTC and CDCP1 positive/keratin negative cells as mCTC.
